(19) United States
(12) Patent Application Publication
(10) Pub. No.: US 2011/0087133 A1
(43) Pub. Date: Apr. 14, 2011

(54) EXTENDABLE SPECIMEN COLLECTING DEVICE

(76) Inventors: Victoria Jessica CHING, Toronto (CA); Nancy SETO, Toronto (CA)

(21) Appl. No.: 12/761,803

(22) Filed: Apr. 16, 2010

Related U.S. Application Data
(60) Provisional application No. 61/249,893, filed on Oct. 8, 2009.

(51) Int. Cl. A61B 10/02

(52) U.S. Cl. 600/572

(57) ABSTRACT

Disclosed is a specimen collecting device for collecting cell samples. The specimen collecting device has an extendible swab which can be extended and retracted during use. The extendible swab can also be inserted into the handle so that the samples can be stored, at least temporarily.

### CROSS-REFERENCE TO RELATED APPLICATION

This application claims priority to U.S. provisional application No. 61/249,893 filed on Oct. 8, 2009, the teachings of which are hereby incorporated by reference in their entirety.

### FIELD OF THE INVENTION

This invention relates to specimen collecting devices and in particular to extending specimen collecting devices.

### BACKGROUND OF THE INVENTION

Advances in diagnostic technology, particularly molecular diagnostics, have allowed for simpler testing procedures than in the past, which required adequate medical facilities and staff. With these advances, some stages of diagnostic testing can be much simpler for doctors, or even be done by patients themselves, potentially relieving significant problems of the previous constraints.

For example, Papanicolau tests (“pap tests”) are still currently the primary method of screening for cervical cancer (caused by the human papillomavirus, or HPV) in women, but this test presents many problems. One such problem is that the pap test must be performed at a clinic, where a doctor must insert a vaginal speculum while a woman lies on her back, and collect a sample from a small area on the cervix with a long swab. For doctors, finding the cervix can be difficult and sample quality can be compromised. For patients, this invasive experience, coupled with other factors such as inconvenience, anxiety or misinformation, can be very uncomfortable for many women and can lead to irregular screening or avoidance altogether. According to the Canadian National Advisory Committe on Immunization (NACI), 30-40% of Canadian women do not screen regularly for cervical cancer (National Advisory Committee on Immunization (NACI), “Statement on human papilloma virus vaccine,” Canada Communicable Disease Report, Volume 33, Feb. 15, 2008). Another problem is that women in rural and underdeveloped locations do not always have access to the facilities or medical staff necessary for regular pap tests.

Human papillomavirus (HPV), the causative agent of cervical cancer, can now be tested for using molecular diagnostics, which would identify HPV DNA in a vaginal specimen. Unlike the pap test which requires a sample of cells to be taken from a specific site on the cervix, HPV can simply be detected in a sample of vaginal fluid, preferably taken from near the cervix area. Clinical studies have shown that the quality of samples collected by patients themselves can be comparable to physician collected samples, potentially allowing patients to screen themselves.

A similar way of collecting samples from other body sites for diagnostic purposes can also be done with many other diseases including, but not limited to, anal HPV, Gonorrhea, Herpes, Syphilis, Trichomoniasis, and Chlamydia. This type of sample collection will also make reactive testing (similar to a pregnancy test) easier in the future, which is likely to also be applied to HPV, Chlamydia and other diseases.

### SUMMARY OF THE INVENTION

According to one aspect, there is provided a specimen collecting device, comprising: a handle; an insertion piece comprising: a cover defining an interior, the cover configured for removable attachment to the handle at an attachment end; and a swab telescopically positioned in the cover, the swab having a collection end, opposite a body portion; the swab movable between an extended position for collecting samples such that the collection end is distal to the attachment end and a retracted position the such that the body portion is enclosed within the cover; an extending mechanism configured for extending the swab while the attachment end is attached to the handle.

According to another aspect, there is provided a method of collecting biological specimens using the specimen collecting device, comprising: inserting the extending end of the device into an orifice; extending the collection end of the swab from the extending end of the device; obtaining samples with the collection end of the swab; and retracting the collection end of the swab.

### BRIEF DESCRIPTION OF THE DRAWINGS

These and other features of the invention will become more apparent from the following description in which reference is made to the appended drawings wherein:

FIG. 1 is a perspective view of a specimen collecting device;

FIG. 2 is a perspective view of a specimen collecting device with a swab in an extended position;

FIG. 2A is a cut-out enlarged perspective view of the collecting end of a swab;

FIG. 3 is an expanded view of a specimen collecting device;

FIG. 4A is a side elevation view of a swab;

FIG. 4B is a cross-sectional side elevation view of a swab;

FIG. 5A is a perspective view of a swab aligner;

FIG. 5B is a side elevation view of a swab aligner;

FIG. 5C is a cross-sectional side elevation view of a swab aligner;

FIG. 6A is a perspective view of an insertion piece without the swab;

FIG. 6B is a side elevation view of an insertion piece without the swab;

FIG. 6C is a cross-sectional side elevation view of an insertion piece without the swab;

FIG. 7A is a perspective view of a handle;

FIG. 7B is a side elevation view of a handle;

FIG. 7C is a cross-sectional side elevation view of a handle;

FIG. 8A is a side elevation view of a specimen collecting device showing the interior of the handle;

FIG. 8B is a cross-sectional side elevation view of a specimen collecting device;

FIG. 9 is a partially expanded and partially cross-sectional view of the specimen collecting device;

FIG. 10 is a side elevation view showing the specimen collecting device in an example use;

FIG. 11A is a diagram showing the specimen collecting device in an example use;

FIG. 11B is a diagram showing the site of an example use;

FIGS. 12A to 12C are side elevation views showing the specimen collecting device in use;

FIG. 13 is a cross-sectional side elevation view showing the specimen collecting device containing the swab in its storing position;

FIG. 14 is a side elevation view showing the specimen collecting device containing the swab in its storing position; and

FIG. 15 depicts a flowchart for collecting biological specimens using the specimen collecting device.

### DETAILED DESCRIPTION OF THE PREFERRED EMBODIMENTS

A specimen collecting device 100 is disclosed. The specimen collecting device 100 is for collecting and/or storing cell samples or biological fluid samples. The specimen collecting device 100 has an extending swab with a collection end that may be extended and/or retracted using an extending mechanism, as described herein. After a sample is collected using the collection end, the swab may be secured within an interior enclosure of the handle for transportation or storage. The interior enclosure of the handle may be sterilized. In a further aspect, the interior enclosure of the handle may be insulated.

According to one aspect, there is provided: a specimen collecting device, comprising: a handle; an insertion piece comprising: a cover defining an interior, the cover configured for removable attachment to the handle at an attachment end; and a swab telescopically positioned in the cover, the swab having a collection end opposite a body portion; the swab movable between an extended position for collecting samples such that the collection end is distal to the attachment end and a retracted position such that the body portion is enclosed within the cover; an extending mechanism configured for extending the swab when engaged.

Generally, the specimen collecting device 100 may be used for the collection of biological specimens such as by a patient or doctor for diagnostic purposes. The device 100 includes an extendable swab and an interior enclosure for storage and/or transportation. In one example, the device 100 can be used to obtain a vaginal fluid sample from the cervix area of a woman for human papillomavirus testing. In another example, the device 100 may similarly be used for testing for other diseases such as anal human papillomavirus, Chlamydia, Gonorrhea, Herpes, Syphilis, Trichomoniasis and other diseases. Further, the device 100 may also be adapted for immediate reactive testing to the specimen for tests such as, but not limited to, Human papilloma virus, Colon cancer, Gonorrhea, or Chlamydia.

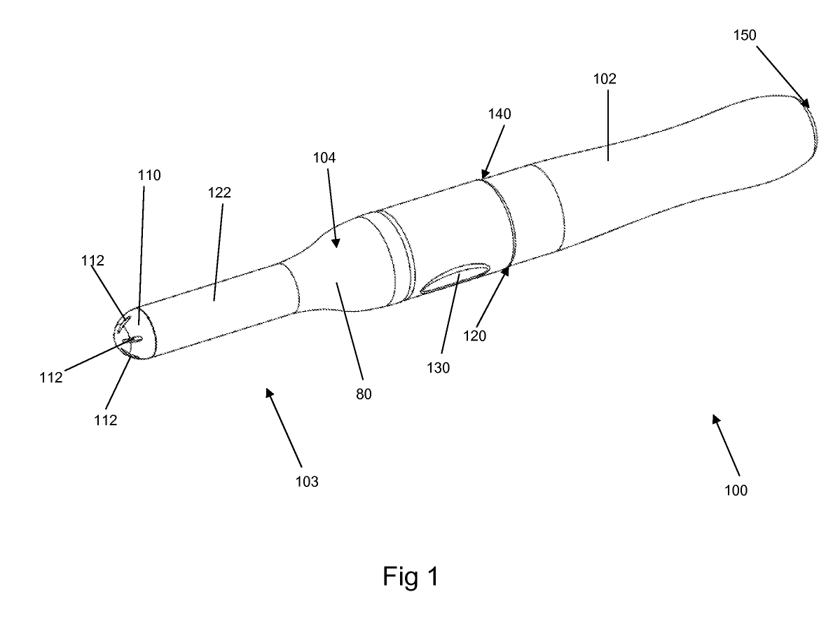

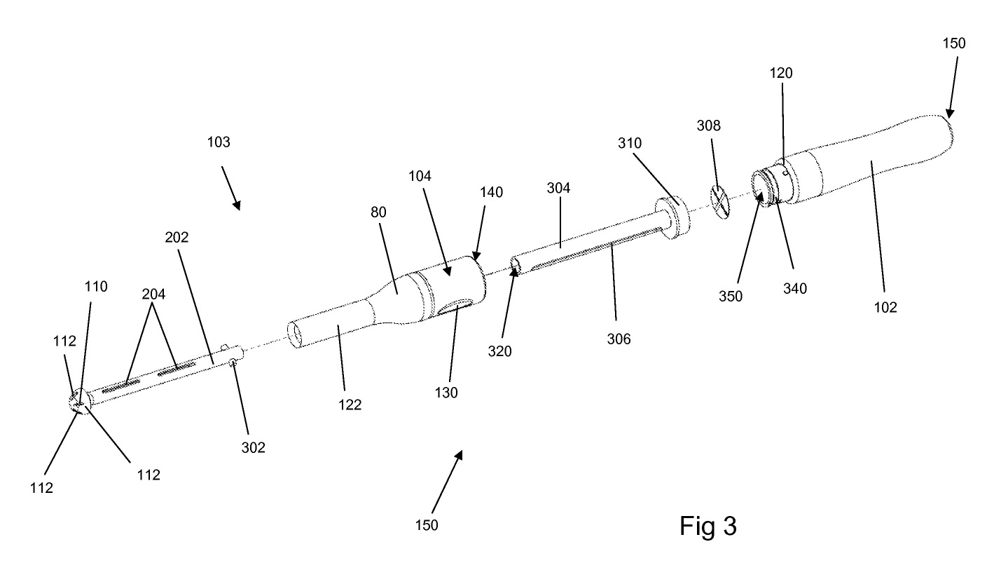

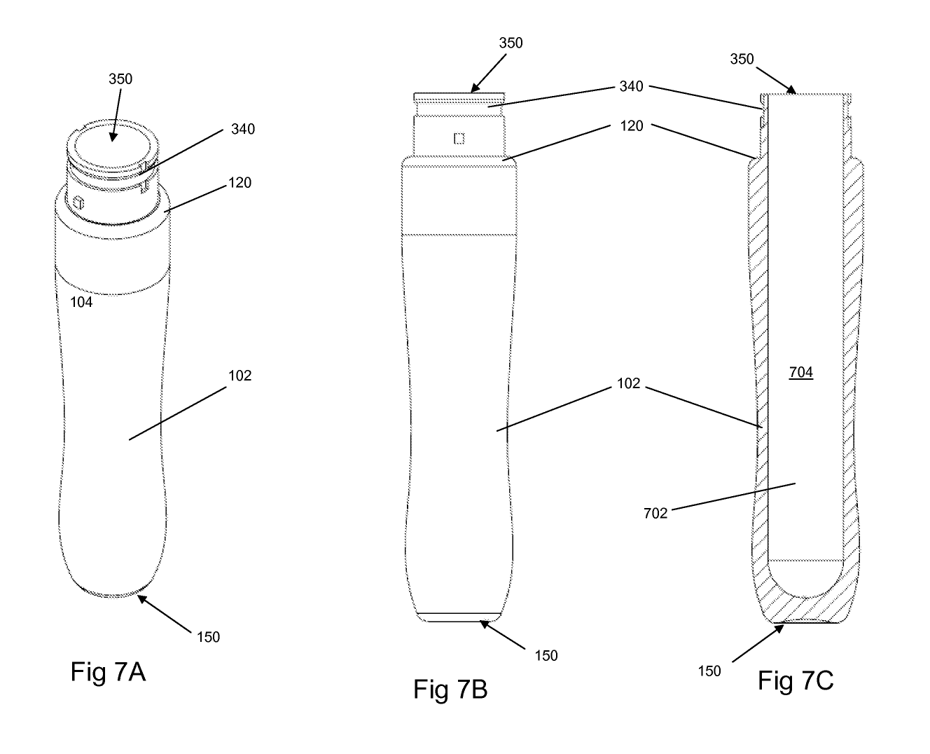

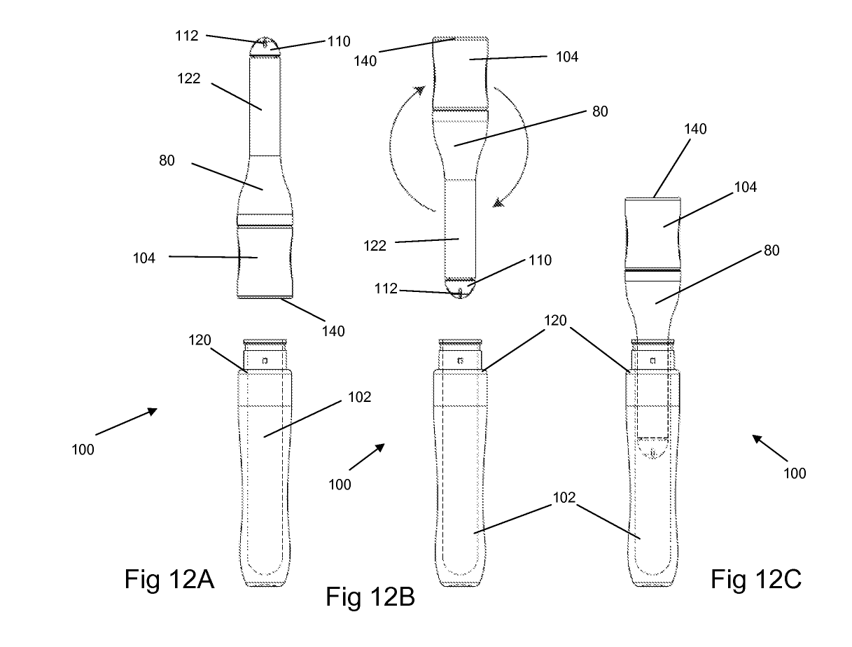

Referring to FIG. 1, the specimen collecting device 100 has a handle 102 and an insertion piece 103. The insertion piece 103 comprises a cover 104, which may be removably attached to an attachment end 120 of the handle 102. As will be described in reference to FIGS. 12A-12C, according to one embodiment, the insertion piece 103 is removably attached to the handle 102 such that when detached from the handle 102, at least a portion of the insertion piece 103 (i.e. at least a portion of the swab of the insertion piece 103) may be housed within an interior channel of the handle 102. In this manner, once the insertion piece 103 is detached from the handle 102, the swab 202 is configured to be received within the handle 102. This allows safe storage and transportation of the swab 202 and the collection end 110 by minimizing contamination of the collected specimen. For example, the cover 104. For example, the cover 104 may be attached by a screw-thread fit, a snap-fit, frictional fit, or another detent fitting mechanism. The cover 104 may have a textured gripping section 130 as markers for holding the insertion piece 103. The textured section 130 may include for example, two dents located on opposing sides of the insertion piece 103 that may be used as markers for the user to place their fingers thereon. FIG. 1 illustrates an exemplary shape of the gripping section 130 but other shapes and configuration of the gripping section 130 may be envisaged by a person skilled in the art. In another example, the textured gripping section 130 may form a band around the circumference of the cover 104. The cover 104 may have an extending end 122 opposite the attachment end 140. In one embodiment, the cover 104 may taper from a larger cross-sectional area at the attachment end 140 to a smaller cross-sectional area at the extending end 122. In another embodiment, the cover 104 may have a constant width and have a cylindrical shape. In yet another embodiment, the cover 104 may have an oval shape in cross-section. In yet a further embodiment, the cover 104 may have various curves and grooves for example to accommodate holding the cover 104 and use of the insertion piece 103 for collection of a sample. In one aspect, the handle 102 may be cylindrical, for example, and may have an open end 350 (FIG. 3) leading into an interior enclosure 702 (FIG. 7C). In another aspect, the handle 102 may have various curves and grooves as may be desired to allow for example, better gripping or holding of the handle 102. Other shapes and contours of the handle 102 and the cover 104 may be envisaged as understood by a person skilled in the art. For example, the handle 102 may have one or more portions that are rectangular, triangular, oval, assymetrical, round with one side flat, or other shapes and including various textures. The cover 104 may also have a central portion 80 made out of a deformable material.

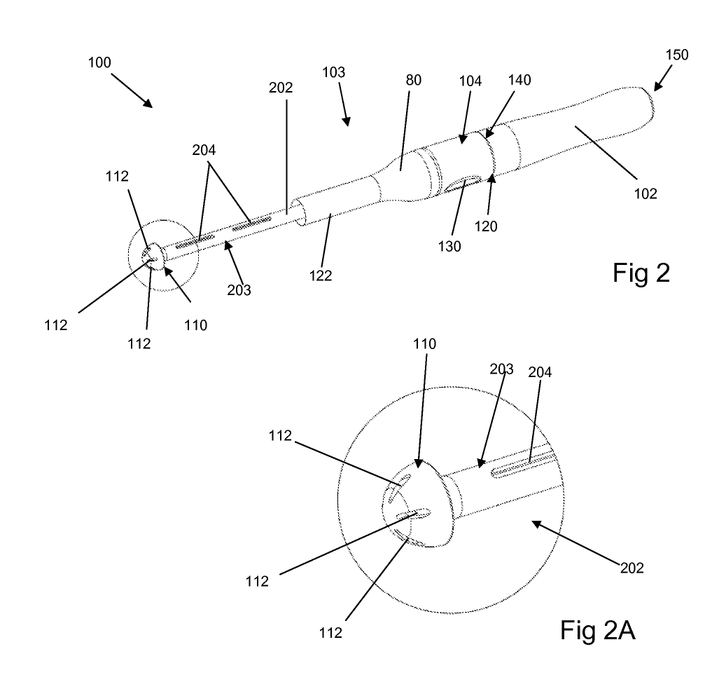

The insertion piece 103 further comprises a swab 202 (FIG. 2). The swab 202 may be at least partially enclosed or positioned in the interior defined by the cover 104. Further, the swab 202 may be in a telescopic engagement with the cover's 104 interior. The swab 202 may be extendable from the extending end 122 of the cover 104 between an extended position (shown in FIG. 2) and a retracted position (shown in FIG. 1). As shown in FIGS. 2 and 2A, the swab 202 comprises a collection end 110 at one end and a body portion 203 at an opposed end. In the retracted position, the body portion 203 of the swab 202 is at least partially located within the cover 104 and proximal to the attachment end 140. Further, in the retracted position, the collection end 110 is located proximal to the extending end 122 of the cover 104. In the extended position, the swab 202 may be extended out of the cover 104 so that the insertion piece 103 is elongated and so that the collection end 110 is distal to the cover 104. That is, in the extended position, the body portion 203 extends outwardly away from the cover 104 such that the body portion 203 is located proximal to the extending end 122 and the collection end 110 is located distal to the extending end 122. Thus, in the extended position, the length of the insertion piece 103 (i.e. the length from the collection end 110 of the swab 202 to the attachment end 140 of the cover 104) is longer than the length of the insertion piece 103 in the retracted position. An extension mechanism (described below) may be used to extend the swab 202 while the attachment end 120 of the handle 102 is attached to the insertion piece 103. The swab 202 may be cylindrical having two grooves 204 running along its surface. Alternatively, the swab 202 may be of made of various shapes and sizes. In one example, the swab 202 may be formed of a different material than the grooves 204. That is, the swab 202 may be made of one or more of the following: a fibrous absorbent material (cotton, Dacron, polyester, or other plastic fibres), bristles (Dacron bristles, polyester bristles, and other plastic bristles) or a sponge material (natural sponge, synthetic sponge, urethane foam, other foam/sponge materials.

At one end of the swab 202 opposite the handle 102 is the collection end 110. The collection end 110 may have a series of grooves 112 for collecting cell samples and/or biological fluid samples. Alternatively, the collection end 110 may have only one groove 112. By way of further alternative, the collection end 110 may comprise an absorbent material for absorbing fluid samples. In one aspect, the collection end 110 may have a number of shapes provided that the collection end 110 abuts against the cover 104 and does not move into the interior of the cover 104. In one example, the collection end 110 may have an arcuate shape and at least one curved portion. For example, the collection end 110 may be any variation of a rounded tip, a half circle, a half oval or different half parabola shapes. In another example, the collection end could also be a cone shaped tip, a flat tip, a tip with a slice through it, a star shape, multi-sided, a circular shape, a twisted shape, or other shapes as envisaged by a person skilled in the art.

The collection end 110 may be made of for example any one of plastic, cardboard, metal, a fibrous absorbent material (cotton, Dacron, polyester, other plastic fibres), made of bristles (Dacron bristles, polyester bristles, other plastic bristles), a sponge material (natural sponge, synthetic sponge, urethane foam, other foam/sponge materials) or a flexible material (silicone, natural rubber, synthetic rubber, other flexible plastics).

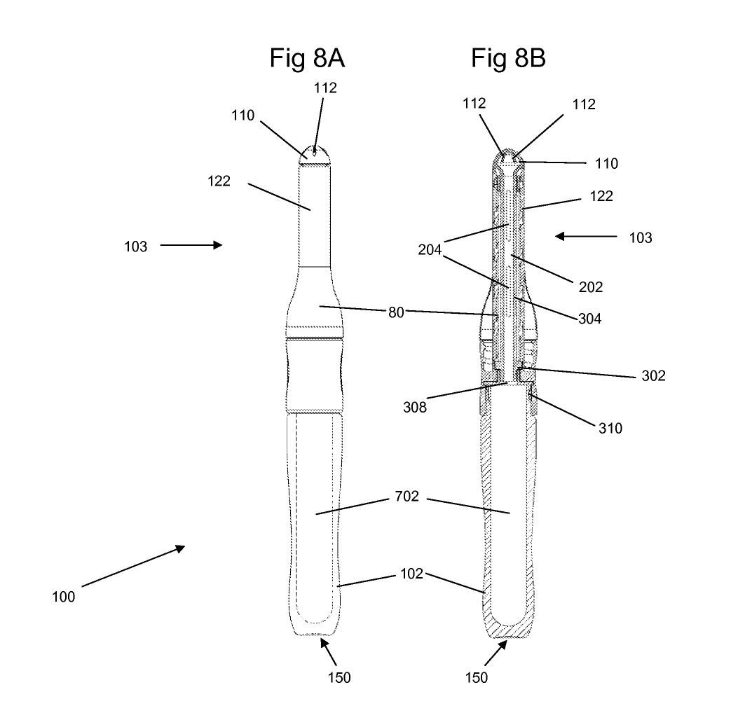

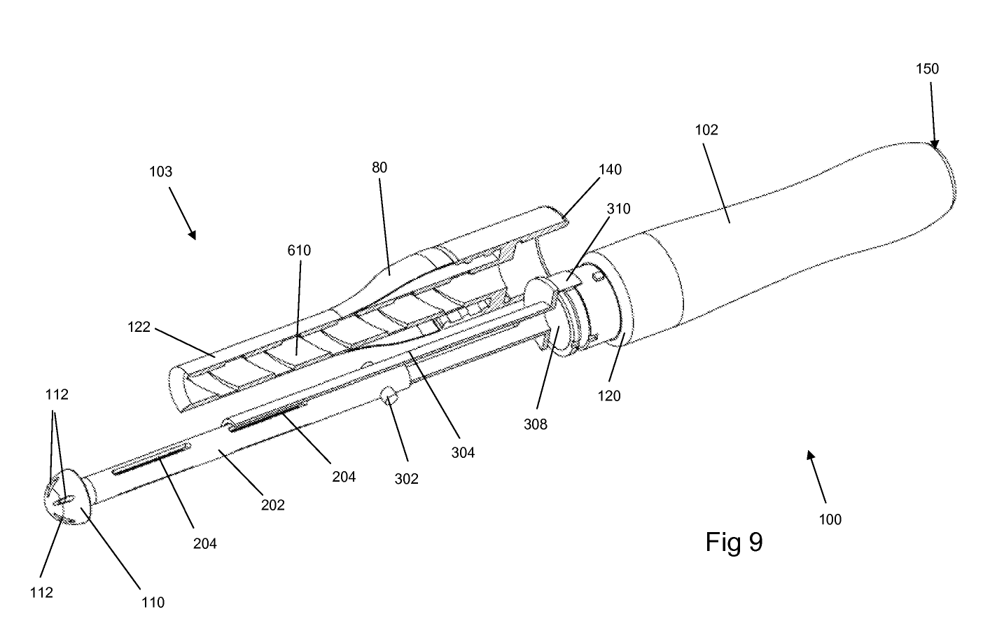

An exploded view of the specimen collection device 100 is shown in FIG. 3. The swab 202 may have an alignment bar 302 near the opposite end of the swab 202 from the collection end 110. A swab aligner 304 may also be enclosed in the cover 104. The swab aligner 304 may have one or more grooves 306 for receiving the alignment bar 302 inside of the cover 104. The swab aligner 304 may be cylindrical and may be in telescoping engagement with the swab 202 inside of the interior (FIG. 8B) of the insertion piece 103 and/or cover 104. Alternatively, the swab aligner 304 may be of a different shape, such as rectangular or another non-symmetrical shape. The swab aligner 304 may have a handle end 310 proximal to the attachment end 140 of the cover 104. A stopper 308 (FIG. 3) may be positioned opposite the handle end 310 from the swab aligner 304. The stopper 308 abuts the open end 350 of the handle 102 when the attachment end 140 of the cover 104 is attached to the handle 102 thus ensuring that no debris or materials enter into the interior enclosure 702 (FIG. 7) of the handle 102.

The attachment end 140 of the cover 104 may, for example, removably attach to a complementary section 340 over the open end 350 of the handle 102 using a screw-thread connection, a snap-fit connection, a frictional fit connection, a detent fitting connection or other connections. Other attachment mechanisms for attaching the attachment end 140 of the cover 104 to the handle 102 over the open end 350 of the handle 102 will be familiar to a person of ordinary skill in the art. The attachment between the attachment end 140 of the cover 104 and the handle 102 may comprise a seal, for example.

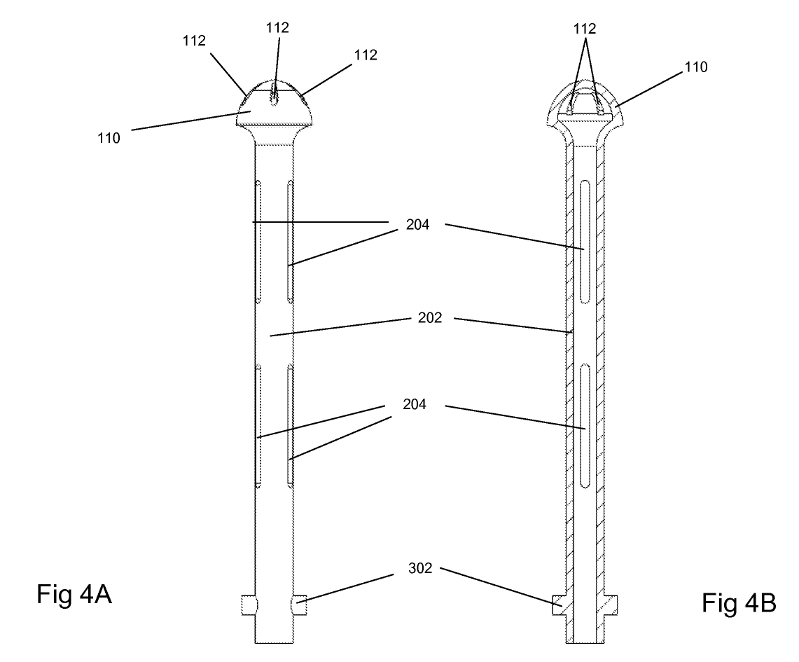

The swab 202 is shown in more detail in FIGS. 4A and 4B. The swab 202 may be hollow, although this is not necessary. The embodiment shown in the figures includes six grooves 204 passing through the swab 202 into its interior and four grooves 112 on the collection end 110 of the swab. However, different numbers and designs of grooves may be used with the present invention. As noted above, the collection end 110 or the entire swab 202 could be made of different materials or have a different collection mechanism from those described, which would be familiar to a person of ordinary skill in the art. The alignment bar 302, which may be at the opposite end of the swab 202 from the collection end 110, may be a separate piece of material which may be inserted into apertures in the swab 202. Alternatively, the alignment bar 302 may be integral with the swab 202. By way of further alternative, the alignment bar 302 may be integral with the swab 202, but on a live hinge that allows it to bend up and down and move in a number of angles relative to the handle 102. The alignment bar 302 may have a number of shapes. For example, it may have a flat surface, a pointy end, or other shapes may be envisaged. There may also be a number of protrusions and grooves on the alignment bar.

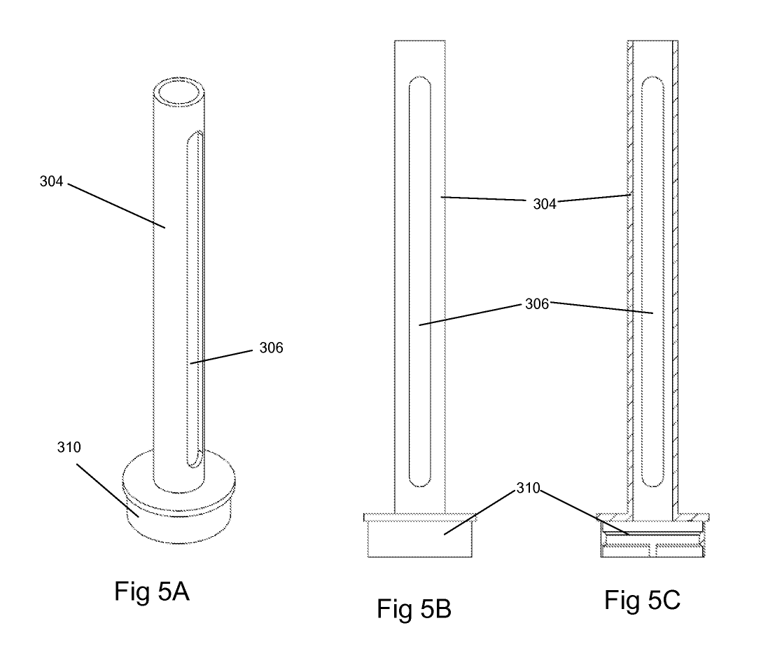

The swab aligner 304 is shown in more detail in FIGS. 5A to 5C. The swab aligner 304 may be a hollow, substantially cylindrical member. It is recognized that the swab aligner 304 may be in a different shape, such as rectangular. However, the swab aligner 304 is preferably shaped to receive the swab 202 in a telescoping relationship so that the swab 202 can slide (at least partially) into and out of the swab aligner 304. The swab aligner 304 may have an opening 320 (FIG. 3) at one end for receiving the swab 202. The swab aligner 304 may be closed at the end opposite the opening 320 or may be abutted by the stopper 308 at the end opposite the opening 320.

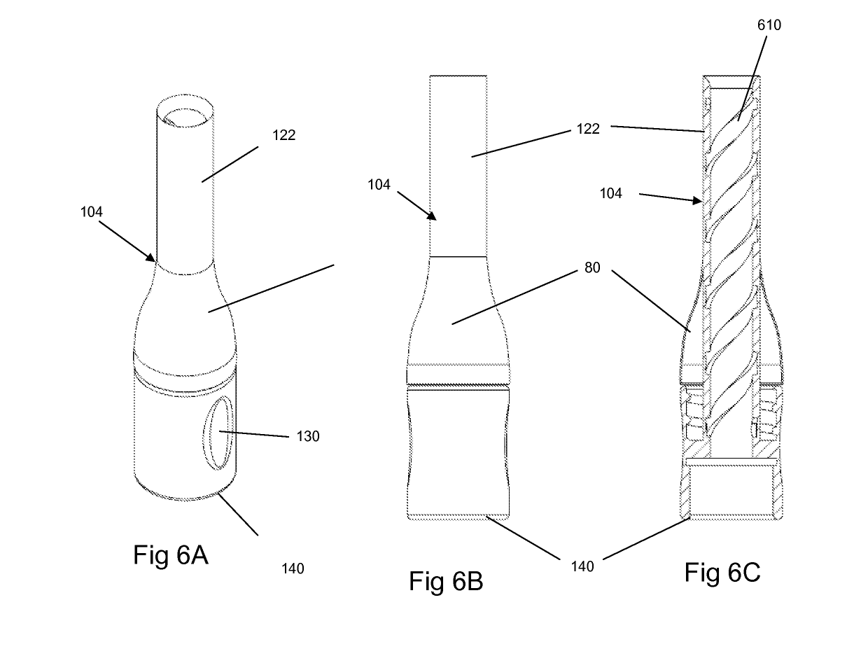

The cover 104 is shown in more detail in FIGS. 6A to 6C. Referring to FIG. 6C, the interior surface of the cover 104 may be threaded with threads 610 sized to fit the alignment bar 302. The threads 610 may run the entire length of the inner surface of the cover 104. Alternatively, the threads 610 may only run partially along the inner surface of the cover 104. Preferably the number of threads is such that the swab is fully extended out of the cover 104 after between one to three rotations. In an alternate embodiment, there are at least two separate threads on the inside of the cover 104 (not shown) such that one thread connects to one side of the alignment bar 302 and another thread connects to an opposing side of the alignment bar 302.

FIGS. 7A to 7C show the handle 102 in more detail. The handle 102 defines an interior 702 with an open end 350 opposite a closed end 150. The closed end 150 of the handle may have a planar exterior surface so that the specimen collection device 100 can rest on a planar surface on its closed end 150. Alternatively, there may be a separate stand (not shown) for holding the specimen collection device 100. The interior 702 may be for storing samples, such as cell samples or other fluid samples, and may run between the open end 350 and the closed end 150. The open end 350 may be the only opening into the interior 702 of the handle 102. The interior surface 704 of the interior 702 may be sterile. Further, the interior enclosure 702 of the handle 102 may be insulated.

When the swab 202 is in the retracted position, shown in FIGS. 8A and 8B for example, the collection end 110 is proximal the extending end 122 of the cover 104. Additionally, the alignment bar 302 may be proximal the attachment end 140 of the cover 104 when the swab 202 is in the retracted position.

Referring to FIGS. 3 and 8B, the alignment bar 302 extends laterally from the swab 202. The swab 202 may be enclosed in a telescopic relationship with the swab aligner 304 so that the alignment bar 302 engages or extends through the grooves 306 in the swab aligner 304. The alignment bar 302 may be engaged with the grooves 306 such that the alignment bar 302 restricts the swab from sliding completely out of the swab aligner 304. For example, the alignment bar 302 may abut the end of at least one of the grooves 306 so that the swab 202 cannot slide any further outward from the swab aligner 304.

The swab aligner 304 (and the swab 202 enclosed therein) may be enclosed in the cover 104 of the insertion piece 103 such that the handle end 310 of the swab aligner 304 is proximal the attachment end 120 of the handle 102. Further, the swab aligner 304 may be freely rotatable within the cover 104. When the swab aligner 304 and swab 202 are enclosed in the cover 104, the alignment bar 302 of the swab 202 may extend through the groove(s) 306 and engage with the thread 610 on the inner surface of the cover 104.

The collection end 110 of the swab 302 may be exterior to the swab aligner 304 when the swab 202 is in a telescopic relationship with the swab aligner 304. In the embodiment shown, the collection end 110 has an arcuate shape such as a mushroom shape which extends outwardly and laterally from the remainder of the swab 202. As discussed earlier, in other embodiments, the collection end 110 may have other shapes providing the size of the collection end 110 is larger than the opening of the swab aligner 304 such as to prevent the collection end 110 from entering the interior of the swab aligner 304. Thus, when the swab 202 is enclosed in the swab aligner 304 (and in the retracted position), the collection end 110 may abut the swab aligner 304 preventing the collection end 110 from telescoping or entering into the interior of the swab aligner 304.

The handle 102 may be rotatably attached to the insertion piece 103. For example, the handle 102 may be rotatably engaged to the cover 104 such that the handle 102 rotates along its longitudinal axis relative to the cover 104. This rotation may be achieved using a bearing(s) or a groove-mechanism as would be familiar to a person of ordinary skill in the art. The attachment of the handle 102 to the cover 104 may be a screw-thread connection, a snap-fit connection, a frictional fit connection, a detent fitting connection or other attachment. The handle 102 may also be secured to the swab aligner 304 so that when the handle 102 rotates relative to the cover 104, the swab aligner 304 also rotates relative to the cover 104. Further, when the swab aligner 304 rotates relative to the cover 104, the alignment bar 302 (and hence, swab 202) are forced to rotate by virtue of the alignment bar's 302 engagement with the groove(s) 306 in the swab aligner 304. Further, when the swab 202 and the alignment bar 302 rotate, the threads 610 are engaged and the alignment bar 302 travels along the threads 610 forcing the swab 202 to move longitudinally in relation to the cover 104. In other words, the when the handle 102 is rotated the swab 202 extends or retracts depending on the direction of rotation. In an alternative embodiment, the extension and retraction movement of the swab 202 may be in a sliding motion such that once triggered using the extension mechanism, the swab 202 slides up or down as needed.

### Operation

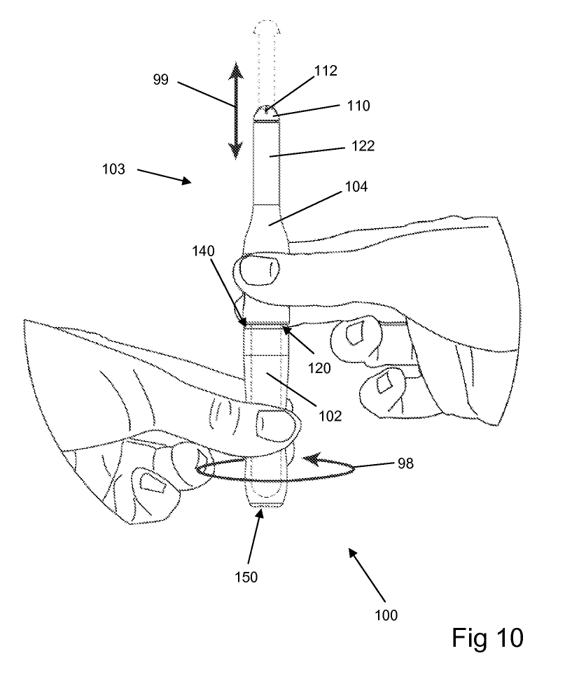

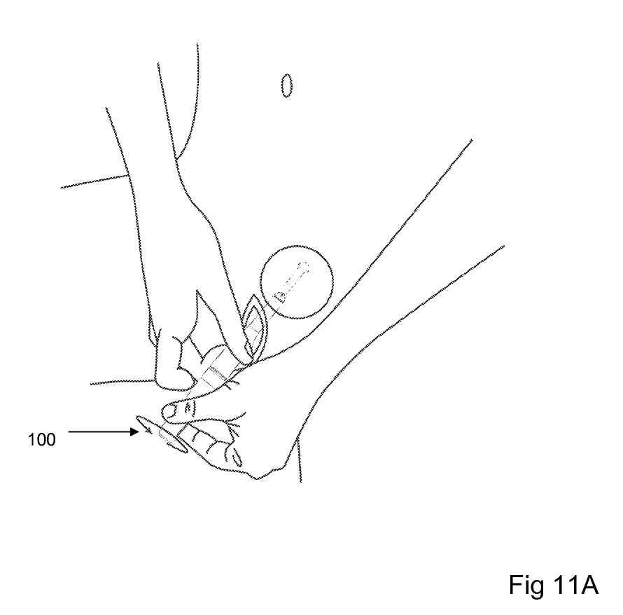

In one embodiment, the operation of the swab 202 is as follows. In operation, the swab 202 is operable to move between the retracted position (shown in FIG. 1) and the extended position (shown in FIG. 2) by rotating the handle 102 in relation to the cover 104. In the present embodiment, the extending mechanism is a rotation mechanism configured to allow the swab 202 to move between the retracted position and the extended position. This rotation may be accomplished by holding the cover 104 with one hand and rotating 98 the handle 102 with the other hand (see FIG. 10). The movement of the swab 202 as a result of the rotation 98 is shown at arrow 99. In alternate embodiments the handle may be activated such as by pushing, pulling and/or squeezing to cause the swab 202 to extend or retract as needed.

As noted, when the handle 102 is rotated in relation to the cover 104 (shown at arrow 98), the swab aligner 304 is also rotated (by virtue of being attached to the handle). When the swab aligner 304 is rotated, the swab 202 is also rotated by virtue of the alignment bar 302 being engaged with the swab aligner 304. When the swab 202 rotates, the alignment bar 302 engages with the threads 610 in the interior of the cover 104. During the rotation of the swab 202 the threads 610 apply a longitudinal force on the alignment bar 302 so that the alignment bar 302 moves along the grooves 603 in the swab aligner 304. As the alignment bar 302 moves along the grooves 603 in the swab aligner 304, the swab 202 itself will slide longitudinally along the swab aligner 304 (i.e. between the extracted position and the retracted position, and vice versa).

Other extensions mechanisms configured for extending and retracting the collection end 110 and/or the swab 202 as envisioned by a person skilled in the art may be used. The extension mechanism may be engaged or triggered (i.e. by twisting the handle 102 or pushing a button as a trigger) to cause the extension or retraction of the swab 202 relative to the handle 102. For example, a hydraulic pump, a battery powered motor, an air pump, etc. may be used. Further, the extension mechanism may be operated by a button located on the handle 102 causing the movement of the swab 202 (i.e. the body portion 203 and the collection end 110) between the retracted position and the extended position shown respectively in FIGS. 1 and 2.

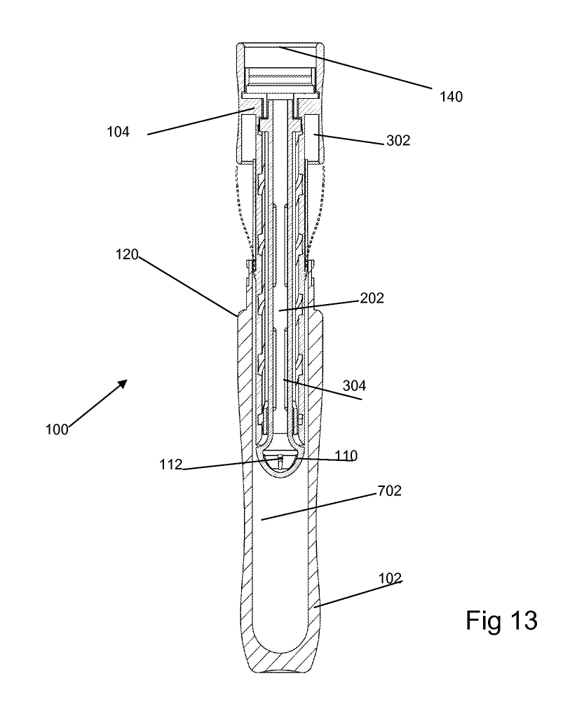

Referring to FIGS. 12A to 12C, the sample taken on the collection end 110 may be stored (at least temporarily) in the handle 102. The insertion piece 103 may be removed from the handle 102 revealing the interior 702 of the handle 102. The insertion piece 103 may be inserted into interior 702 of the handle 102 such that the collection end 110 is inside of the interior 702 of the handle 102, as shown in FIG. 13. This allows for example for safe storage and subsequent transport of the sample on the collection end 110. When the insertion piece 103 is inserted into the interior 702 of the handle 102, the extending end 122 of the cover 104 may be sealably and removably attached over the opening 350 of the handle 102.

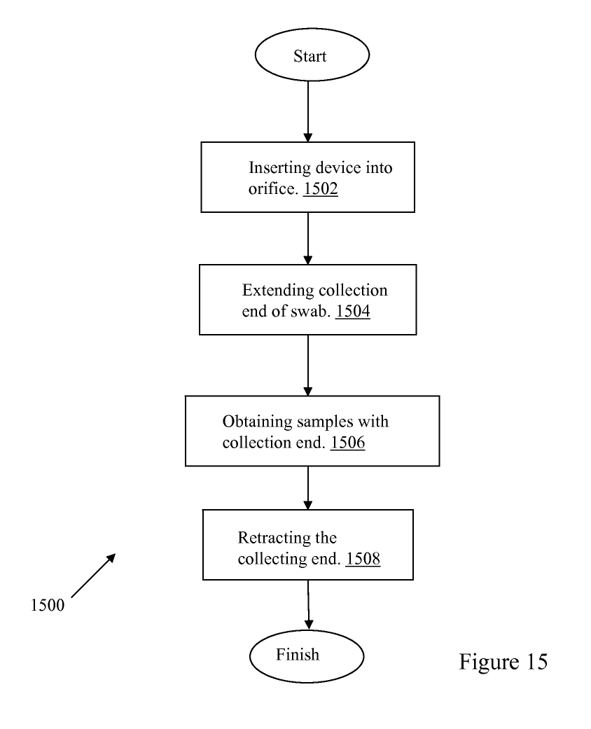

A method for collecting biological specimens using the specimen collecting device 100 is shown generally at FIG. 15. At step 1502 the extending end 122 of the device 100 is inserted into a suitable orifice of a person (e.g a vagina or an anus). At step 1504 the collection end 110 of the swab 202 is extended from the extending end 122 of the device 100. This may be accomplished using the mechanism for extending the collection end 110 of the swab 202 or the mechanism for extending the swab 202, described above. At step 1506, samples are obtained with the collection end 110 of the swab 202. Finally, at step 1508 the collection end 110 of the swab 202 is retracted. The device 100 may then be removed. Further, at step 1510, the insertion end 103 may be removed from the handle 102 and the collection end 110 may be inserted into the interior enclosure 702 of the handle 102 for storage and/or transportation.

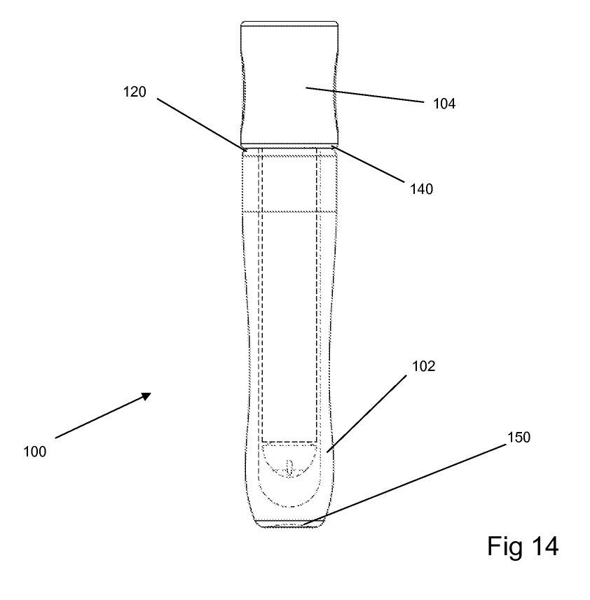

Referring to FIGS. 13 and 14, when the extending end 122 of the cover 104 (and the collection end 110 of the swab 202) is pushed into the interior 702 of the handle 102, the central portion 80 of the cover may be deformed so that a seal may be formed between the cover 104 and the handle 102. Alternatively, the cover 104 may be attached to the handle 102 using a screw, snap-fit, frictional fit, detent connection, or other attachment mechanism familiar to persons of ordinary skill in the art. The attachment between the extending end 122 of the cover and the handle 102 may or may not be sealable. It is recognized that a seal between the handle 102 and the cover 104 may be formed by other means familiar to skilled persons.

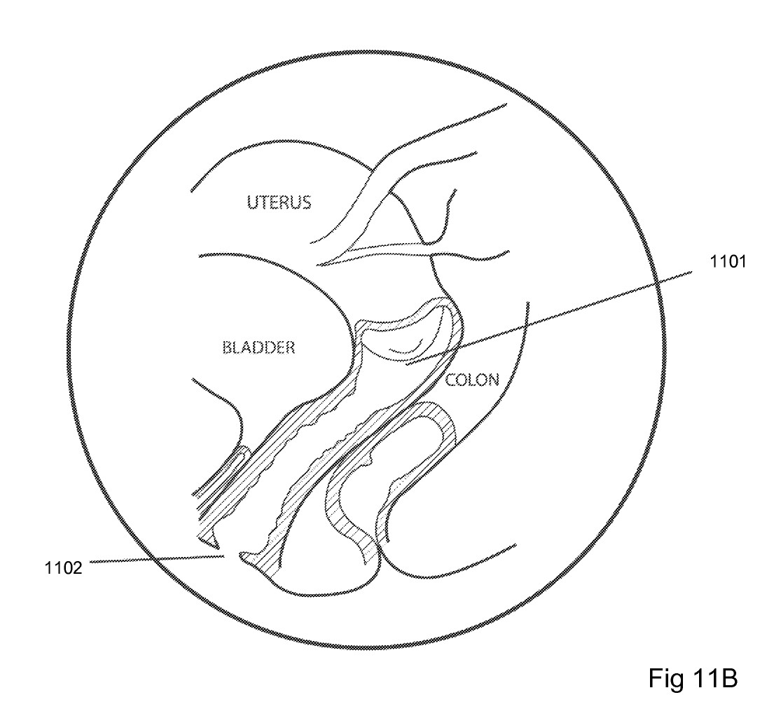

According to an embodiment, when the swab 202 is in the retracted position, the insertion piece 103 may be approximately 80 mm to 105 mm in length (i.e. between the collection end of the swab and the attachment end 140 of the cover 104). Further, when the swab 202 is in the extended position, the insertion piece 103 may be approximately 100-145 mm in length. Generally, when the swab 202 is in the extended position, it should be long enough to reach from the introitus to the cervix 1102 or cervix area 1101 (FIG. 11B) while the handle 102 is gripped by the patient, or a doctor or a nurse (see FIG. 11A, for example).

The width of the extending end 122 (or diameter of the extending end 122 in the case of a cylindrical extending end 122) is preferably sufficiently wide for the extension and retraction of a structurally sound swab 202, but should not exceed a width which may be visually or physically uncomfortable to a patient, such as approximately 18-20 mm. The length of the handle 102 is preferably sufficiently long for comfortable handling, such as approximately 80 mm. The interior 702 of the handle 102 is preferably wide enough and deep enough to house the extending end 122 of the insertion piece 103. For example, the interior 702 may have a depth of 77 mm.

Generally, the size of the specimen collecting device 100 may be sized to accommodate for mailing, transportation and storage in a test tube rack, such as approximately 20 mm wide at its widest part.

It is understood that other, alternative lengths, widths and sizes may be used depending on the embodiment of the specimen collecting device 100.

The construction details of the specimen collecting device 100 may be as follows. The swab 202 and extending end 122 of the insertion piece 103 may be made of a biodegradable plastic or any other sufficiently rigid and strong plastic, cardboard or other material. The portion of the inner swab 202 that is enclosed in the cover 104 when the swab 202 is in the retracted position may be covered or coated with a another material such as Dacron®. The area of the cover 104 between the attachment end 140 and the central portion 80 may be made out of or coated with rubber or another textured material. The handle 102 may preferably be made of transparent or semi-transparent biodegradable plastic, or any other sufficiently rigid and strong transparent or semi-transparent plastic. It is understood that other materials may also be used for the specimen collecting device 100.

One or more currently preferred embodiments have been described by way of example. It will be apparent to persons skilled in the art that a number of variations and modifications can be made without departing from the scope of the invention as defined in the claims.

What is claimed is the following:

1. A specimen collecting device, comprising:
a handle;
an insertion piece comprising:
a cover defining an interior, the cover configured for removable attachment to the handle at an attachment end; and
a swab telescopically positioned in the cover, the swab having a collection end opposite a body portion; the swab movable between an extended position for collecting samples such that the collection end is distal to the attachment end and a retracted position such that the body portion is enclosed within the cover;
an extending mechanism configured for extending the swab when engaged.

2. The specimen collecting device of claim 1, wherein the cover comprises an extending end opposite the attachment end of the cover such that in the retracted position the collection end is located proximal to the extending end.

3. The specimen collecting device of claim 2, wherein the extending mechanism is configured for extending the body portion outwardly and away from the extending end when the swab is extended from the retracted position to the extended position.

4. The specimen collecting device of claim 1 wherein the extending mechanism is further configured for retracting the swab relative to the handle when the extending mechanism is engaged.

5. The specimen collecting device of claim 4 wherein the extending mechanism is configured for at least one of retracting and extending the swab while the attachment end of the cover is attached to the handle.

6. The specimen collecting device of claim 1, wherein the handle is rotatably attachable to the cover, and rotating the handle relative to the cover when attached to the cover applies a longitudinal force onto the swab relative to the cover, wherein the extending mechanism for extending the swab comprises a mechanism adapted to rotate the handle relative to the cover when the handle is attached to the cover.

7. The specimen collecting device of claim 1, wherein the extending mechanism comprises one of a hydraulic pump, a battery operated pump, and an air-pressure pump.

8. The specimen collecting device of claim 7, wherein the extending mechanism for extending the swab is activated by a button attached to the handle.

9. The specimen collecting device of claim 1 wherein the handle comprises an interior enclosure defining a channel extending therethrough and open at one end thereof, the channel configured for receiving at least a portion of the cover and the swab when the attachment end is detached from the handle.

10. The specimen collecting device of claim 1 wherein the handle comprises an interior enclosure and one open end leading to the interior enclosure, the extending end of the cover removably attachable over the open end of the handle for storing the collecting end in the interior enclosure of the handle for storing collected samples from collecting end of the device in the interior enclosure of the handle.

11. The specimen collecting device of claim 10, wherein the attachment of the cover over the open end of the handle forms a seal for restricting at least one fluid and gas from exiting the interior enclosure of the handle.

12. The specimen collecting device of claim 10, the cover further comprises a deformable material, wherein the deformable material is deformed when the extending end of the cover is attached over the open end of the handle thereby forming the seal.

13. The specimen collecting device of claim 1, the collection end comprises a series of grooves for collecting specimen samples.

14. The specimen collecting device of claim 1, the collection end comprises absorbent material for absorbing specimen samples.

15. The specimen collecting device of claim 1, wherein the interior enclosure of the handle is insulated.

16. The specimen collecting device of claim 3, the interior enclosure of the handle having a sterile interior surface.

17. A method of collecting biological specimens using the specimen collecting device of claim 1, comprising:
inserting the extending end of the device into an orifice;
extending the collection end of the swab from the extending end of the device;
obtaining samples with the collection end of the swab; and
retracting the collection end of the swab.

18. A method of collecting biological specimens using the specimen collecting device of claim 10, comprising:
inserting the extending end of the device into an orifice;
extending the collection end of the swab from the extending end of the device;
obtaining samples with the collection end of the swab;
retracting the collection end of the swab; and
inserting the collection end of the device into the interior enclosure of the handle.

19. A specimen collecting device assembly, the assembly comprising:
a handle; and
an insertion piece adapted for removable connection to the handle, the insertion piece comprising a cover and a swab located within an interior of the cover, the swab having a collection end adapted to collect the specimen;
wherein when the insertion piece is connected to the handle, the swab is adapted to move between an extended position for collecting samples such that a body portion of the swab is extended telescopically out of the cover and the collection end of the swab is distal to the handle, and a refracted position such that the body portion is enclosed within the cover.

20. The specimen collecting device assembly of claim 19 wherein the handle comprises a hollow interior configured for receiving the insertion piece for subsequent storage of the insertion piece and collection end when the insertion piece is detached from the handle.
